SEQUENCE LISTING (52 sequences)

SEQ ID NO: 1 - DNA, 21 bp, Artificial Sequence
  Description of Artificial Sequence Synthetic primer
tgaggaaccctacgaagaagc

SEQ ID NO: 2 - DNA, 20 bp, Artificial Sequence
  Description of Artificial Sequence Synthetic primer
cgagatacttgtcaacggca

SEQ ID NO: 3 - DNA, 20 bp, Artificial Sequence
  Description of Artificial Sequence Synthetic primer
tttcaacctccaagagacgc

SEQ ID NO: 4 - DNA, 21 bp, Artificial Sequence
  Description of Artificial Sequence Synthetic primer
aagacgcaggagtactctccc

SEQ ID NO: 5 - DNA, 20 bp, Artificial Sequence
  Description of Artificial Sequence Synthetic primer
accaccacacaatcaaagcg

SEQ ID NO: 6 - DNA, 20 bp, Artificial Sequence
  Description of Artificial Sequence Synthetic primer
ttacgacgtttccgaagagg

SEQ ID NO: 7 - DNA, 20 bp, Artificial Sequence
  Description of Artificial Sequence Synthetic primer
tggacgaatgtcagcagaac

SEQ ID NO: 8 - DNA, 20 bp, Artificial Sequence
  Description of Artificial Sequence Synthetic primer
tgtatgaacccacggtgttg

SEQ ID NO: 9 - DNA, 22 bp, Artificial Sequence
  Description of Artificial Sequence Synthetic primer
ggcattgtgattggagtagtgg

SEQ ID NO: 10 - DNA, 22 bp, Artificial Sequence
  Description of Artificial Sequence Synthetic primer
cttgttaggtgggtcattggag

SEQ ID NO: 11 - DNA, 20 bp, Artificial Sequence
  Description of Artificial Sequence Synthetic primer
cacagagaacgacaacaccg

SEQ ID NO: 12 - DNA, 20 bp, Artificial Sequence
  Description of Artificial Sequence Synthetic primer
cgaatgactccctcaccagt

SEQ ID NO: 13 - DNA, 20 bp, Artificial Sequence
  Description of Artificial Sequence Synthetic primer
ttcgcagaacctactcaggc

SEQ ID NO: 14 - DNA, 21 bp, Artificial Sequence
  Description of Artificial Sequence Synthetic primer
gccaacacagatacgatgagc

SEQ ID NO: 15 - DNA, 20 bp, Artificial Sequence
  Description of Artificial Sequence Synthetic primer
ctcggacagccaacaattca

SEQ ID NO: 16 - DNA, 22 bp, Artificial Sequence
  Description of Artificial Sequence Synthetic primer
ccactcctgcctttctacactt

SEQ ID NO: 17 - DNA, 22 bp, Artificial Sequence
  Description of Artificial Sequence Synthetic primer
caccaccatccttaccatcatc

SEQ ID NO: 18 - DNA, 21 bp, Artificial Sequence
  Description of Artificial Sequence Synthetic primer
agaatgatgagcaagcacagc

SEQ ID NO: 19 - DNA, 24 bp, Artificial Sequence
  Description of Artificial Sequence Synthetic primer
catgataaccctcaaatatgtccc

SEQ ID NO: 20 - DNA, 19 bp, Artificial Sequence
  Description of Artificial Sequence Synthetic primer
gcccagtgtaggctggagt

SEQ ID NO: 21 - DNA, 20 bp, Artificial Sequence
  Description of Artificial Sequence Synthetic primer
gtgatggtgaacgggatcct

SEQ ID NO: 22 - DNA, 18 bp, Artificial Sequence
  Description of Artificial Sequence Synthetic primer
gttggcaggccacacgcc

SEQ ID NO: 23 - DNA, 21 bp, Artificial Sequence
  Description of Artificial Sequence Synthetic primer
agccagaagatatcaccgagg

SEQ ID NO: 24 - DNA, 21 bp, Artificial Sequence
  Description of Artificial Sequence Synthetic primer
tcagtccagaactcgtcttcg

SEQ ID NO: 25 - DNA, 20 bp, Artificial Sequence
  Description of Artificial Sequence Synthetic primer
gtttactggtgatcgctgcc

SEQ ID NO: 26 - DNA, 22 bp, Artificial Sequence
  Description of Artificial Sequence Synthetic primer
tgggctgtggaagtatagtgac

SEQ ID NO: 27 - DNA, 22 bp, Artificial Sequence
  Description of Artificial Sequence Synthetic primer
ctacagcagccaaaacgagaag

SEQ ID NO: 28 - DNA, 24 bp, Artificial Sequence
  Description of Artificial Sequence Synthetic primer
cctggcctggtaacaagtatactc

SEQ ID NO: 29 - DNA, 22 bp, Artificial Sequence
  Description of Artificial Sequence Synthetic primer
gtgaggttagaggagcatttgg

SEQ ID NO: 30 - DNA, 20 bp, Artificial Sequence
  Description of Artificial Sequence Synthetic primer
caggaatgtaacacgccatg

SEQ ID NO: 31 - DNA, 22 bp, Artificial Sequence
  Description of Artificial Sequence Synthetic primer
ctggcaaacaaaagaattggac

SEQ ID NO: 32 - DNA, 20 bp, Artificial Sequence
  Description of Artificial Sequence Synthetic primer
agatccgcaaagcagtctcc

SEQ ID NO: 33 - DNA, 21 bp, Artificial Sequence
  Description of Artificial Sequence Synthetic primer
atgtcctcaaccagctacacg

SEQ ID NO: 34 - DNA, 20 bp, Artificial Sequence
  Description of Artificial Sequence Synthetic primer
cttaaccttcccagccttcc

SEQ ID NO: 35 - DNA, 57 bp, Homo sapiens
tgtcccaaagtttactcaagactacccaggaacctcttgcccgagatcctgttaaac

SEQ ID NO: 36 - DNA, 157 bp, Homo sapiens
ccggcacagtcttcactaccgtagaagaccttggctccaagatactcctcacctgctccttgaatgacagcgccacagaggtcacagggcaccgctggctgaaggggggcgtggtgctgaaggaggacgcgctgcccggccagaaaacggagttcaa

SEQ ID NO: 37 - DNA, 147 bp, Homo sapiens
ctgtgatgaaggctacattggagcaaggtgtgagagagttgacttgttttacctaagaggagacagaggacagattctggtgatttgtttgatagcagttatggtagtttttattattttggtcatcggtgtctgcacatgctgtca

SEQ ID NO: 38 - DNA, 147 bp, Homo sapiens
atgtgaatgaatgcacctccggacaaaacccgtgccacagctccacccactgcctcaacaacgtgggcagctatcagtgccgctgccgcccgggctggcaaccgattccggggtcccccaatggcccaaacaataccgtctgtgaag

SEQ ID NO: 39 - DNA, 53 bp, Homo sapiens
ggcaagcgaccagcgtgatctcacagagcacaaaccctcagtctccaaccaca

SEQ ID NO: 40 - DNA, 30 bp, Homo sapiens
agagttttgaggtgacagtcaccaaagaag

SEQ ID NO: 41 - DNA, 159 bp, Homo sapiens
gcactaactgtgacatctatgaccttttttatcatcgcacaagcccctgaaccatatattgttatcactggatttgaagtcaccgttatcttatttttcatacttttatatgtactcagacttgatcgattaatgaagtggttattttggcctttgctt

SEQ ID NO: 42 - DNA, 160 bp, Homo sapiens
aaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagag

SEQ ID NO: 43 - DNA, 54 bp, Homo sapiens
gccttactcagttgcccaattccgcagaagaactggacagtgaggacctctcag

SEQ ID NO: 44 - DNA, 84 bp, Homo sapiens
attccagagtcagtgtcacaaaaccatttatgttaccccctgttgcagccagctcccttaggaatgacagcagtagcagtaata

SEQ ID NO: 45 - DNA, 96 bp, Homo sapiens
aacccccgcacagtccctgttcagcctgccttctccacggtgccgttctcccagcctgtctgtttcccacccaggcccagggggcgcagacaaaaa

SEQ ID NO: 46 - DNA, 347 bp, Homo sapiens
agcttttcaggaagcatctgaacttccagtctcaggtcagctggatgatgccacaagggcccgcatgaggcagcctcgttgtggcctagaggatcccttcaaccagaagacccttaaatacctgttgctgggccgctggagaaagaagcacctgactttccgcatcttgaacctgccctccacccttccaccccacacagcccgggcagccctgcgtcaagccttccaggactggagcaatgtggctcccttgaccttccaagaggtgcaggctggtgcggctgacatccgcctctccttccatggccgccaaagctcgtactgttccaatacttttgatgggcctg

SEQ ID NO: 47 - DNA, 24 bp, Homo sapiens
agcatcttgggattgaatttatgg

SEQ ID NO: 48 - DNA, 321 bp, Homo sapiens
gtgatttctattcactactttccaagctgctaggagaaagggaagatgttgttcatgtgcacaaatataatcctacagaaaaggcagaatcagagtcagacctggtagctgagattgcaaatgtagtccaaaaaaaggatcttggtcgatctgatgccagagagggtgcagaacatgagaggggtaatgctattcttgtcagagacagaattcacaaattccacagactagtatctaccttgaggccaccagagagcagagttttctcattacagcagcccccacctggtgaaggcacctgggaaccagaacacacaggag

SEQ ID NO: 49 - DNA, 69 bp, Homo sapiens
gaaggcctagggagtcaggtaaaaaacggaaaagaaaaaggttaaaacccacctaaagcagccaaccag

SEQ ID NO: 50 - DNA, 24 bp, Homo sapiens
ctaaagaacagtatttcattccag

SEQ ID NO: 51 - DNA, 54 bp, Homo sapiens
gcagtttcagcgtcgaaagtggccagaaatgaagagaccttcttccaaatcact

SEQ ID NO: 52 - DNA, 21 bp, Artificial Sequence
  Description of Artificial Sequence Synthetic oligonucleotide
ttttttttttttttttttttt

(19) United States
(12) Patent Application Publication
(10) Pub. No.: US 2012/0122113 A1
(43) Pub. Date: May 17, 2012

(54) Signature Of Secreted Protein Isoforms Specific To Ovarian Cancer

(75) Inventors: Panagiotis Prions, Sherbrooke (CA); Julian Venables, Sherbrooke (CA); Benoit Chabot, Sherbrooke (CA); Sherif Abou Elela, Stoke (CA)

(73) Assignee: SOCPRA SCIENCES SANTE ET HUMAINES S.E.C., Sherbrooke, QC (CA)

(21) Appl. No.: 13/128,955

(22) PCT Filed: Apr. 14, 2010
(86) PCT No.: PCT/CA2010/000570

§ 371 Date: Dec. 19, 2011

Related U.S. Application Data
(60) Provisional application No. 61/169,000, filed on Apr. 14, 2009.

(51) Int. Cl. C12Q 1/68; G01N 33/573; G01N 33/566

(52) U.S. Cl. 435/6.18; 436/501; 435/7.4

(57) ABSTRACT

The present invention relates to the identification of secreted protein isoforms specific in ovarian cancer and methods for diagnosis or prognosis of ovarian cancer in a subject by detecting the secreted protein isoforms.

### TECHNICAL FIELD

The present disclosure is directed to secreted protein isoforms specifically expressed in ovarian cancer, to the signature these isoforms make as well as to methods for diagnosis or prognosis of ovarian cancer in a subject by detecting these secreted protein isoforms.

### BACKGROUND ART

The transformation of a normal cell into a malignant cell results, among other things, in the uncontrolled proliferation of the progeny cells, which exhibit immature, undifferentiated morphology, exaggerated survival, proangiogenic properties, expression, overexpression or constitutive activation of oncogenes not normally expressed in this form by normal, mature cells.

Nearly all cancers are caused by abnormalities in the genetic material of the transformed cells. These abnormalities may be due to the effects of carcinogens, such as tobacco smoke, radiation, chemicals, or infectious agents. Other cancer-promoting genetic abnormalities may be randomly acquired through errors in DNA replication, or are inherited, and thus present in all cells from birth. Complex interactions between carcinogens and the host genome may explain why only some develop cancer after exposure to a known carcinogen. New aspects of the genetics of cancer pathogenesis, such as DNA methylation, and microRNAs are increasingly being recognized as important.

One example of cancer is epithelial ovarian cancer which is the second most common gynaecological cancer and the deadliest amongst gynaecological pelvic malignancies. Early symptoms of ovarian cancer are often mild and unspecific, making this disease difficult to detect. In most cases, at the time of diagnosis, cancer cells have already disseminated throughout the peritoneal cavity. In fact, over 70% of patients are diagnosed with late stage disease and only a minority survive over 5 years post-diagnosis. Early detection offers a 90% 5-year survival rate. The inability to detect ovarian cancer at an early stage and its propensity for peritoneal metastasis are largely responsible for these low survival rates.

Currently, there are no reliable methods for detecting early stages of epithelial ovarian cancer. Blood level of CA125 tumour antigen is employed as a predictor of clinical recurrence of ovarian cancers, and to monitor response to anticancer therapy (Yang et al., 1994, Zhonghua Fu Chan Ke Za Zhi, 29: 147-149). The CA125 serum marker combined with transvaginal ultrasonography are the current clinical tests offered for screening for early stages of ovarian cancer in high risk populations (Nikolic et al., 2006, Bosn J Basic Med Sci 6: 3-6). However, neither of these modalities individually or combined have proven reliable (Nikolic et al., 2006, Bosn J Basic Med Sci 6: 3-6), and there is an urgent need to develop new screening tests to detect epithelial ovarian cancer at an early stage.

Epithelial ovarian tumours are heterogeneous and include many different histopathological subtypes: serous, endometrioid, mucinous, clear cell, undifferentiated or mixed. The serous type is the most frequent and the second most lethal. Recent studies have focused on differences in molecular profiling of gene expression patterns to uncover diagnostic and prognostic markers as well as new therapeutic targets in a variety of cancers. Although promising results have been reported in some cancers, the genes that are differentially expressed between normal and cancer cells seem to vary between individual microarray studies, reflecting either a variability in methods and in the choice of model systems or a heterogeneity in selected tissues (Kopper & Timar, 2005, Pathol Oncol Res, 11: 197-203).

Detection of many cancers still relies on detection of an abnormal mass in the organ of interest. In many cases, a tumor is often detected only after a malignancy is advanced and may have metastasized to other organs. Thus, there is a need for methods for earlier detection of ovarian cancer. Such new methods could, for example, replace or complement the existing ones, reducing the margins of uncertainty and expanding the basis for medical decision making.

It would be highly desirable to be provided with novel biomarkers for the early detection, prognosis and clinical management of ovarian cancer. Sensitive and specific tests that can diagnose different stages of ovarian cancer would greatly improve patient survival rates by facilitating early diagnosis and tailored therapies. It would also be highly desirable to be provided with new screening tests to detect ovarian cancer at an early stage.

### SUMMARY

The present disclosure relates to a method for diagnosis or prognosis of ovarian cancer in a subject by detecting a signature of at least one secreted protein isoform. The method comprises the steps of obtaining a sample from the subject, and determining whether the sample contains the signature specific to ovarian cancer. The presence of the signature is indicative of the presence of ovarian cancer in the individual or the progression of ovarian cancer in the individual.

It is also provided a diagnostic or prognostic kit for ovarian cancer for detecting a signature of at least one secreted protein isoform in a sample of a subject, the kit comprising a moiety that specifically recognized the protein isoform or a mRNA encoding the protein isoform of the signature and a set of instructions for using the moiety to detect the signature specific to ovarian cancer.

In a further embodiment, it is provided a method for profiling an ovarian cancer in a subject by detecting a signature of at least one secreted protein isoform comprising obtaining a sample from the subject and determining whether the sample contains the signature specific to ovarian cancer.

The signature may comprise at least two secreted protein isoforms.

In a further embodiment, the secreted protein isoform are selected from amyloid beta A4 protein precursor (APP), basigin precursor (BSG), probetacellulin precursor (BTC), CD97 antigen precursor (CD97), Carcinoembryonic antigen-related cell adhesion molecule 1 precursor (CEACAM1), Calsyntenin-1 precursor (CLSTN1), Chemokine-like factor (CMTM1 or CKLF), Tissue factor precursor (F3), Cell adhesion molecule 1 precursor (IGSF4), Kit ligand precursor (KITLG), Galectin-9 (LGSF9), Matrix metalloproteinase-19 precursor (MMP19), Neuregulin-1 (NRG1), Peptidyl-glycine alpha-amidating monooxygenase precursor (PAM), Platelet-derived growth factor A chain precursor (PDGFA), Extracellular sulfatase Sulf-2 precursor (SULF-2), and/or Slit homolog 2 protein precursor (SLIT2).

In another embodiment, In a further embodiment, the secreted protein isoform is amyloid beta A4 protein precursor (APP), basigin precursor (BSG), probetacellulin precursor (BTC), CD97 antigen precursor (CD97), Carcinoembryonic antigen-related cell adhesion molecule 1 precursor (CEACAM1), Calsyntenin-1 precursor (CLSTN1), Chemokine-like factor (CMTM1 or CKLF), Tissue factor precursor (F3), Cell adhesion molecule 1 precursor (IGSF4), Kit ligand precursor (KITLG), Galectin-9 (LGSF9), Matrix metalloproteinase-19 precursor (MMP19), Neuregulin-1 (NRG1), Peptidyl-glycine alpha-amidating monooxygenase precursor (PAM), Platelet-derived growth factor A chain precursor (PDGFA), Extracellular sulfatase Sulf-2 precursor (SULF-2), or Slit homolog 2 protein precursor (SLIT2), or any combination thereof.

In another embodiment, the APP secreted splice variant is encoded by the nucleotide sequence set forth in SEQ ID NO:35; the BSG secreted splice variant is encoded by the nucleotide sequence set forth in SEQ ID NO:36; the BTC secreted splice variant is encoded by the nucleotide sequence set forth in SEQ ID NO:37; the CD97 secreted splice variant is encoded by the nucleotide sequence set forth in SEQ ID NO:38; the CEACAM1 secreted splice variant is encoded by the nucleotide sequence set forth in SEQ ID NO:39; the CLSTN1 secreted splice variant is encoded by the nucleotide sequence set forth in SEQ ID NO:40; the CMTM1 or CKLF secreted splice variant is encoded by the nucleotide sequence set forth in SEQ ID NO:41; the F3 secreted splice variant is encoded by the nucleotide sequence set forth in SEQ ID NO:42; the IGSF4 secreted splice variant is encoded by the nucleotide sequence set forth in SEQ ID NO:43; the KITLG secreted splice variant is encoded by the nucleotide sequence set forth in SEQ ID NO:44; the LGSF9 secreted splice variant is encoded by the nucleotide sequence set forth in SEQ ID NO:45; the MMP19 secreted splice variant is encoded by the nucleotide sequence set forth in SEQ ID NO:46; the NRG1 secreted splice variant is encoded by the nucleotide sequence set forth in SEQ ID NO:47; the PAM secreted splice variant is encoded by the nucleotide sequence set forth in SEQ ID NO:48; the PDGFA secreted splice variant is encoded by the nucleotide sequence set forth in SEQ ID NO:49; the SLIT2 secreted splice variant is encoded by the nucleotide sequence set forth in SEQ ID NO:50; or the SULF-2 secreted splice variant is encoded by the nucleotide sequence set forth in SEQ ID NO: 51.

### BRIEF DESCRIPTION OF THE DRAWINGS

Having thus generally described the nature of the invention, reference will now be made to the accompanying drawings.

FIG. 1 illustrates histogram representations of the splicing patterns of identified secreted proteins isoforms (A: APP; B: BSG; C: BTC; D: CD97; E: CEACAM1; F: CLSTN1; G: CMTM1 (or CKLF); H: F3; I: IGSF4; J: KITLG; K: LGALS9; L: MMP19; M: NRG1; N: PAM; 0: PDGFA; P: SULF2; Q: SLIT2), the histograms representing bins of ψ values of 5 with the smoothened distribution as a dashed line.

FIG. 2 illustrates the detection by ELISA of secreted protein isoform F3 in patient sera with ovarian cancer (OVC) compared to a normal patient (N).

### DETAILED DESCRIPTION

In accordance with the present disclosure, there is provided the identification of secreted protein isoforms specific for ovarian cancer. Because these isoforms are advantageously predicted to be secreted, they can easily be detected in a biological fluid of a subject.

The new secreted or transmembrane protein isoforms disclosed herein were identified as secreted products of alternatively spliced mRNA enriched in ovarian cancer specimens. These secreted or transmembrane protein isoforms form a very unique signature specific for ovarian cancer.

Alternative splicing of pre-mRNA is a post-transcriptional process that allows the production of distinct mRNAs from a single gene with the potential to expand protein structure and diversity. Alternative splicing can also introduce or remove regulatory elements to affect mRNA translation, localization or stability. More than 70% of human genes may undergo alternative splicing with many genes capable of producing dozens and even hundreds of different isoforms.

In multicellular organisms, alternative splicing is a process that is tightly regulated during development and in different tissues. Inherited and acquired changes in pre-mRNA splicing patterns have been associated with several human diseases including cancer (Venables, 2006, Bioessays, 28: 378-386). Some of these changes can arise from mutations at either the splice sites or within proximal splicing enhancer or silencer elements (Pagani & Baralle, 2004, Nat Rev Genet, 5: 389-396). In other cases, variations in the expression of trans-acting splicing factors have been observed (Brinkman, 2004, Clin Biochem, 37: 584-594). A direct effect in splice site used, resulting in the production of cancer-specific protein isoforms, has been observed in a few cases (Karni et al., 2007, Struct Mol Biol, 14: 185-193). Cancer-specific alterations in splice site selection can affect genes controlling cellular proliferation (e.g., FGFR2, p53, MDM2, FHIT and BRCA1), cellular invasion (e.g., CD44, Ron), angiogenesis (e.g, VEGF), apoptosis (e.g, Fas, Bcl-x and caspase-2) and multidrug resistance (e.g., MRP-1).

The term “variant” refers to all types of RNAs transcribed from a given gene that, when processed collectively, encode plural protein isoforms. The term “alternative splicing” refers to all types of RNA processing that lead to the expression of plural protein isoforms from a single gene. Some genes, usually eukaryotic genes, are first transcribed as long mRNA precursors that are then shortened by a series of processing steps to produce the mature mRNA molecule. One of these steps is RNA splicing, in which the intron sequences are removed from the mRNA precursor. A cell can splice the primary transcript in different ways, making different “splice variants” and thereby making different polypeptide chains from the same gene, or from the same mRNA molecule. Splice variants can include, for example, exon insertions, exon extensions, exon truncations, exon deletions, alternatives in the 5′ untranslated region and alternatives in the 3′ untranslated region. Splicing variants are unpredictable since the cell can splice the primary transcript in different ways, making different polypeptide chains from the same gene, or from the same mRNA molecule.

The term “protein isoform” refer to any of several forms of a protein encoded by a given gene. In the context of this application, the “protein isoform” are resulting from alternative splicing of a mRNA transcribed from a given gene. Because the protein isoform described herewith are specific to ovarian cancer, the alternative splicing of the mRNAs is modulated at the onset of ovarian cancer or during the progression of ovarian cancer and results in the expression (and secretion) of the protein isoforms. In addition to the differential alternative splicing of mRNAs resulting in the expression (and secretion) of the proteins isoforms, the protein isoforms described herein can also include other isoforms such as those due to the presence of a mutation (such as a SNP) or a different glycosylation pattern.

The molecular etiology of cancer involves genetic predispositions affecting the pathway deregulation, self sufficiency in growth signals, angiogenesis, a deregulated cell cycle and evasion of apoptosis. Genes can also favour changes in cell adhesion and migration properties, local invasion, motility and attachment to distant organ sites. This requires drastic changes and complex rewiring in cellular architecture including cytoskeletal plasticity and epithelial-mesenchymal transition. Alternative splicing is ideally suited to contribute to such complex cellular processes due to its subtle, adaptable and reversible nature. Alternative splicing can contribute to tumorigenesis by producing isoforms that stimulate cell proliferation, cell migration or that induce resistance to apoptosis and anticancer agents. Cancer associated splicing shifts may occur through mutations affecting the splice sites or nearby control elements, but also by alterations in the expression of proteins that control splicing decisions.

Following initial computational efforts designed to exploit collections of expressed sequence tag (EST) databases, there has been an increase in high-throughput experimental approaches to identify changes in splicing events under a variety of conditions. Oligonucleotide-based microarray technologies have been introduced to identify global alternative splicing events and to examine changes in the alternative splicing of a large collection of events. These approaches are useful for monitoring the expression of known splice variants. However, they are not designed to discover novel splice sites, nor do they provide information on the combinatorial patterns of exon inclusion/skipping in the same gene. Furthermore, the lack of standardized analysis and normalization can compromise the interpretation of the results.

Arrays made from alternative splice junction probes have been used to detect splicing changes in Hodgkin Lymphoma (Relogio et al., 2005, J Biol Chem, 280: 4779-4784) and breast cancer cell lines and xenografts (Li et al., 2006, Cancer Res, 66: 1990-1999). A related medium-throughput technique has been used to show that alternative splicing analysis can complement the power of gene expression analysis of prostate tumours (Li et al., 2006, Cancer Res, 66: 4079-4088; Zhang et al., 2006, BMC Bioinformatics, 7: 202). So far, 30 different genes have been shown to be alternatively spliced in a cancer-specific manner (Venables, 2004, Cancer Res, 64: 7647-7654). In ovarian tumours specifically, three genes have been reported to be regulated at the level of splicing (He at al., 2007, Oncogene, advance online publication, Feb. 19, 2007; Sigalas et al., 1996, Nat Med 2: 912-917).

It is provided herein that a layered and integrated system for splicing isoform annotation platform (LISA) was used to identify novel splicing variants resulting in secreted or transmembrane protein isoforms specific to ovarian cancer. The LISA platform relies on automated RT-PCR technology that generates tissue-specific annotation of alternative splicing events as disclosed in International application publication No. WO 09/021,338, the content of which is enclosed herewith by reference. The bioinformatics infrastructure supporting the annotation effort helps assess the potential functional impact of individual alternative splicing events and allows adaptable visualization of large sets of validated results.

The LISA platform was used to identify enriched splice variants in ovarian cancer. Because cancer associated splicing shifts may altered the expression of proteins, the LISA platform was further used to identify secreted or transmembrane protein isoforms enriched in ovarian cancer specimens. There is reported herein a set of highly significant and biologically differences in the expression of secreted protein isoforms that make up a strong signature for ovarian cancer samples.

A secreted protein corresponds to a protein which is secreted outside of the cell membrane. Secreted proteins include, but are not limited to, transmembrane proteins which can be secreted in the extracellular milieu as well as those that remain associated with the extracellular side of the cellular membranes. Secreted or transmembrane proteins such as cytokines, chemokines, hormones, digestive enzymes, antibodies as well as components of the extra-cellular matrix, are secreted from cells into the extra-cellular space. They play pivotal biological regulatory roles and have the potential for protein therapeutics. The majority of secreted proteins have a signal peptide according to the signal hypothesis. Signal peptides are located at the N-terminal of nascent proteins and their lengths are usually <70 amino acid residues. They are cleaved during the process of entering the endoplasmic reticulum (ER) lumen. The signal peptide is a hallmark of secreted proteins. However, many transmembrane (TM) proteins also have a signal peptide. Several secreted protein prediction methods have been developed mainly based on the analysis of signal peptides, and genome-wide identification of potential novel secreted proteins has been reported.

The splicing pattern of all simple alternative splicing events (i.e. cassette exons or alternative 5′ or 3′ splice sites) found in the RefSeq database (Pruitt et al., 2007, Nucleic Acids Res, 35: D61-D65) in ovarian tissues was examined. Each of these events gives rise to long and short isoforms that can be monitored in a single RT-PCR reaction. This is done using capillary electrophoresis, which determines the amplicon concentrations relative to an exogenous internal standard. The percent-spliced-in (psi or LP) for each alternative splicing event is calculated by dividing the concentration of the long isoform amplicon by the sum of the concentrations of both products (see Table 1). The alternative splicing patterns were determined using LISA, a high-throughput PCR-based platform.

Once alternative splicing patterns were determined, an implemented and improved secreted protein prediction approach was used to select from the alternative splicing patterns identified which correspond to secreted protein isoforms. Alternative splicing events occurring in secreted or transmembrane proteins can be selected by fetching data from the Uniprot database and using keyword search such as “secreted” or “transmembrane”.

Table 1 disclosed the list of secreted ovarian-cancer alternative splicing variants identified as biomarkers of the signature of ovarian cancer.

TABLE 1
Properties of secreted ovarian cancer-specific
alternative splicing (AS) variants.

| | | Domain affected by | Significance |
|---|---|---|---|
| Gene | Protein fate | splicing | (P-value) |
| Biomarker 1: | Transmembrane/ | transmembrane | 0.0012 |
| APP | secreted | deletion | |
| Biomarker 2: | Transmembrane/ | Juxtamembrane | 0.000294 |
| BSG | secreted | cleavage | |
| Biomarker 3: | Transmembrane/ | Transmembrane | 3.23E−09 |
| BTC | secreted | deletion | |
| Biomarker 4: | membrane/ | Cadherin motif | 3.57e−28 |
| CD97 | secreted | | |
| Biomarker 5: | Transmembrane/ | Endoproteolytic site | 2.53E−09 |
| CEACAM1 | secreted | | |
| Biomarker 6: | secreted | Cell retention signal | 5.4e−8 |
| CLSTN1 | | | |
| Biomarker 7: | Transmembrane/ | C-term frameshift | 2.73E−18 |
| CMTM1 | secreted | | |
| (or CKLF) | | | |
| Biomarker 8: | Transmembrane/ | Transmembrane | 2.71E−05 |
| F3 | secreted | truncation | |
| Biomarker 9: | Transmembrane/ | EGF domain | 1.11E−05 |
| IGSF4 | secreted | | |
| Biomarker 10: | Transmembrane/ | Protease cleavage site | 6.16E−7 |
| KITLG | secreted | | |
| Biomarker 11: | Transmembrane/ | Protease cleavage site | 6.16E−7 |
| LGALS9 | secreted | | |
| Biomarker 12: | Transmembrane | juxtamembrane | 7.20E−06 |
| MMP19 | | | |
| Biomarker 13: | Transmembrane/ | Protease cleavage | 6.13E−14 |
| NRG1 | secreted | | |
| Biomarker 14: | Transmembrane/ | Proteolytic site | 7.38E−05 |
| PAM | secreted | | |
| Biomarker 15: | secreted | EGF motif | 4.66E−05 |
| PDGFA | | | |
| Biomarker 16: | secreted | Linker domain | 1.04E−07 |
| SLIT2 | | | |
| Biomarker 17: | secreted | Linker arm domain | 6.09E−05 |
| SULF2 | | | |

The specific sequence of secreted alternatively spliced variants identified by the LISA platform is disclosed in Table 2.

TABLE 2
Alternative sequence of secreted ovarian cancer variants.

| Name of | |
|---|---|
| biomarker | Alternative or splice variant sequence |
| APP | tgtcccaaag tttactcaagactacccaggaacctcttgcccgagatcctgttaaac (SEQ ID NO: 35) |
| BSG | ccggcacagtcttcactaccgtagaagaccttggctccaagatactcctcacctgctccttgaatgacagcgccacagaggtca |
| | cagggcaccgctggctgaaggggggcgtggtgctgaaggaggacgcgctgcccggccagaaaacggagttcaa (SEQ |
| | ID NO: 36) |
| BTC | ctgtgatgaaggctacattggagcaaggtgtgagagagttgacttgttttacctaagaggagacagaggacagattctggtgatt |
| | tgtttgatagcagttatggtagtttttattattttggtcatcggtgtctgcacatgctgtca (SEQ ID NO: 37) |
| CD97 | atgtgaatgaatgcacctccggacaaaacccgtgccacagctccacccactgcctcaacaacgtgggcagctatcagtgccg |
| | ctgccgcccgggctggcaaccgattccggggtcccccaatggcccaaacaataccgtctgtgaag (SEQ ID NO: 38) |
| CEACAM1 | ggcaagcgaccagcgtgatctcacagagcacaaaccctcagtctccaaccaca (SEQ ID NO: 39) |
| CLSTN1 | agagttttgaggtgacagtcaccaaagaag (SEQ ID NO: 40) |
| CMTM1 (or | gcactaactgtgacatctatgaccttttttatcatcgcacaagcccctgaaccatatattgttatcactggatttgaagtcaccgttat |
| CKLF) | cttatttttcatacttttatatgtactcagacttgatcgattaatgaagtggttattttggcctttgctt (SEQ ID NO: 41) |
| F3 | aaaacagccaaaacaaacactaatgagtttttgattgatgtggataaaggagaaaactactgtttcagtgttcaagcagtgattc |
| | cctcccgaacagttaaccggaagagtacagacagcccggtagagtgtatgggccaggagaaaggggaattcagag |
| | (SEQ ID NO: 42) |
| IGSF4 | gccttactcagttgcccaattccgcagaagaactggacagtgaggacctctcag (SEQ ID NO: 43) |
| KITLG | attccagagtcagtgtcacaaaaccatttatgttaccccctgttgcagccagctcccttaggaatgacagcagtagcagtaata |
| | (SEQ ID NO: 44) |
| LGALS9 | aacccccgcacagtccctgttcagcctgccttctccacggtgccgttctcccagcctgtctgtttcccacccaggcccagggggc |
| | gcagacaaaaa (SEQ ID NO: 45) |
| MMP19 | agcttttcaggaagcatctgaacttccagtctcaggtcagctggatgatgccacaagggcccgcatgagg |
| | cagcctcgttgtggcctagaggatcccttcaaccagaagacccttaaatacctgttgctgggccgctggagaaagaagcacct |
| | gactttccgcatcttgaacctgccctccacccttccaccccacacagcccgggcagccctgcgtcaagccttccaggactgga |
| | gcaatgtggctcccttgaccttccaagaggtgcaggctggtgcggctgacatccgcctctccttccatggccgccaaagctcgta |
| | ctgttccaatacttttgatgggcctg (SEQ ID NO: 46) |
| NRG1 | agcatcttgggattgaatttatgg (SEQ ID NO: 47) |
| PAM | gtgatttctattcactactttccaagctgctaggagaaagggaagatgttgttcatgtgcacaaatataatcctacagaaaaggca |
| | gaatcagagtcagacctggtagctgagattgcaaatgtagtccaaaaaaaggatcttggtcgatctgatgccagagagggtgc |
| | agaacatgagaggggtaatgctattcttgtcagagacagaattcacaaattccacagactagtatctaccttgaggccaccag |
| | agagcagagttttctcattacagcagcccccacctggtgaaggcacctgggaaccaga acacacaggag (SEQ ID |
| | NO: 48) |
| PDGFA | gaaggcctagggagtcaggtaaaaaacggaaaagaaaaaggttaaaacccacctaaagcagccaaccag (SEQ ID |
| | NO: 49) |
| SLIT2 | ctaaagaacagtatttcattccag (SEQ ID NO: 50) |
| SULF2 | gcagtttcagcgtcgaaagtggccagaaatgaagagacct tcttccaaatcact (SEQ ID NO: 51) |

Amyloid beta A4 protein precursor (APP) functions as a cell surface receptor and performs physiological functions on the surface of neurons relevant to neurite growth, neuronal adhesion and axonogenesis. APP is also involved in cell mobility and transcription regulation through protein-protein interactions. It can promote transcription activation through binding to APBB1/Tip60 and inhibit Notch signalling through interaction with Numb. APP also acts as a kinesin 1 membrane receptor, mediating the axonal transport of beta-secretase and presenilin 1. It is involved in copper homeostasis/oxidative stress through copper ion reduction. It was demonstrated that APP can regulate neurite outgrowth through binding to components of the extracellular matrix such as heparin and collagen I and IV. APP is expressed at the cell membrane as a single-pass type I membrane protein. APP is a cell surface protein that rapidly becomes internalized via clathrin-coated pits. During maturation, the immature APP (N-glycosylated in the endoplasmic reticulum) moves to the Golgi complex where complete maturation occurs (O-glycosylated and sulfated). After alpha-secretase cleavage, soluble APP is released into the extracellular space and the C-terminal is internalized to endosomes and lysosomes. Some APP accumulates in secretory transport vesicles leaving the late Golgi compartment and returns to the cell surface. APP is expressed in all fetal tissues examined with highest levels in brain, kidney, heart and spleen. A weak expression was also reported in liver.

Basigin precursor (BSG) plays pivotal roles in spermatogenesis, embryo implantation, neural network formation and tumor progression. BSG stimulates adjacent fibroblasts to produce matrix metalloproteinases (MMPS) and may target monocarboxylate transporters SLC16A1, SLC16A3 and SLC16A8 to plasma membranes of retinal pigment epithelium and neural retina. It also seems that BSG might be a receptor for oligomannosidic glycans. BSG is expressed at the cell membrane as a single-pass type I membrane protein. BSG was identified by mass spectrometry in melanosome fractions from stage I to stage IV. Normally, BSG is only present in vascular endothelium in non-neoplastic regions of the brain, whereas it is present in tumor cells but not in proliferating blood vessels in malignant gliomas. Interestingly, BSG is enriched on the surface of tumor cells and up-regulated in gliomas. BSG expression levels correlate with malignant potential of the tumor.

Probetacellulin precursor (BTC) is a potent mitogen for retinal pigment epithelial cells and vascular smooth muscle cells. The effects of betacellulin are probably mediated by the EGF receptor and other related receptors. BTC is secreted in the extracellular space. BTC is expressed at the cell membrane as a single-pass type I membrane protein. BTC is synthesized in several tissues and tumor cells. It is predominantly expressed in pancreas and small intestine. Betacellulin from beta cells could play a role in the vascular complications associated with diabetes.

CD97 antigen precursor is a receptor potentially involved in both adhesion and signaling processes early after leukocyte activation. It plays an essential role in leukocyte migration. CD97 is a cell membrane protein with multi-pass. CD97 is normally secreted in the extracellular space. It is broadly expressed, found on most hematopoietic cells, including activated lymphocytes, monocytes, macrophages, dendritic cells, and granulocytes. CD97 is expressed also abundantly by smooth muscle cells and in the thyroid, colorectal, gastric, esophageal and pancreatic carcinomas. CD97 expression is increased under inflammatory conditions in the CNS of multiple sclerosis and in synovial tissue of patients with rheumatoid arthritis. Increased expression of CD97 in the synovium is accompanied by detectable levels of soluble CD97 in the synovial fluid.

Carcinoembryonic antigen-related cell adhesion molecule 1 precursor (CEACAM1) is also expressed at the cell membrane as a single-pass type I membrane protein and can also be secreted. Loss or reduced expression of CEACAM1 is a major event in colorectal carcinogenesis.

Calsyntenin-1 precursor (CLSTN1) induces KLC1 association with vesicles and functions as a cargo in axonal anterograde transport. It may modulate calcium-mediated postsynaptic signals. CLSTN1 is an endoplasmic reticulum membrane, single-pass type I membrane protein. It is normally localized at the postsynaptic membrane of both excitatory and inhibitory synapses. It is expressed in the brain and, at a lower level, in the heart, skeletal muscle, kidney and placenta. CLSTN1 accumulates in dystrophic neurites around the amyloid core of Alzheimer disease senile plaques.

Chemokine-like factor (CMTM1 or also known has CKLF) may play an important role in inflammation and regeneration of skeletal muscle. CKLF1 has chemotactic response in rat monocytes, neutrophils, and lymphocytes. CKLF can be secreted or expressed at the cell membrane as a multi-pass membrane protein depending on the isoform. All CKLF isoforms have highest expression levels in adult spleen, lungs, testis, ovary, peripheral blood leucocyte, placenta, pancreas, fetal brain, skeletal muscle, thymus and heart. Lower expression levels were also reported in adult skeletal muscle, liver, thymus colon, prostate, fetal spleen and liver.

Tissue factor precursor (F3) initiates blood coagulation by forming a complex with circulating factor VII or VIIa. The F3:VIIa complex activates factors IX or X by specific limited protolysis. F3 plays a role in normal hemostasis by initiating the cell-surface assembly and propagation of the coagulation protease cascade. It is localized at the membrane as a single-pass type I membrane protein. F3 expression is highly dependent upon cell type. F3 can also be induced by the inflammatory mediators interleukin 1 and TNF, as well as by endotoxin, to appear on monocytes and vascular endothelial cells as a component of cellular immune response.

Cell adhesion molecule 1 precursor (IGSF4) mediates homophilic cell-cell adhesion in a Ca²⁺-independent manner. IGSF4 acts as a tumor suppressor in non-small-cell lung cancer (NSCLC) cells. Interaction with CRTAM promotes natural killer (NK) cell cytotoxicity and interferon-gamma (IFN-gamma) secretion by CD8+ cells in vitro as well as NK cell-mediated rejection of tumors expressing CADM3 in vivo. IGSF4 may contribute to the less invasive phenotypes of lepidic growth tumor cells. In mast cells, IGSF4 may mediate attachment to and promote communication with nerves. IGSF4 is a single-pass type I cell membrane protein. It is localized at the basolateral plasma membrane of epithelial cells in gall bladder. IGSF4 is absent or down-regulated in many advanced cases of NSCLC as well as in many other human cancers, due to gene silencing by promoter methylation.

Kit ligand precursor (KITLG) stimulates the proliferation of mast cells. KITLG augment the proliferation of both myeloid and lymphoid hematopoietic progenitors in bone marrow culture and also mediates cell-cell adhesion. KITLG is single-pass type I cell membrane protein but also exists as a secreted soluble form.

Galectin-9 (LGSF9) binds galactosides. LGSF9 may play a role in thymocyte-epithelial interactions relevant to the biology of the thymus. LGSF9 induces T-helper type 1 lymphocyte (Th1) death. LGSF9 may be found in the cytoplasm but also can be secreted. LGSF9 is expressed in peripheral blood leukocytes and lymphatic tissues and overexpressed in Hodgkin's disease tissue.

Matrix metalloproteinase-19 precursor (MMP19) is an endopeptidase that degrades various components of the extracellular matrix, such as aggrecan and cartilage oligomeric matrix protein during development, haemostasis and pathological conditions (arthritic disease). It may also play a role in neovascularization or angiogenesis. MMP19 is secreted at the extracellular space. MMP19 is expressed in mammary gland, placenta, lung, pancreas, ovary, small intestine, spleen, thymus, prostate, testis colon, heart and blood vessel walls. MMP19 is also expressed in the synovial fluid of normal and rheumatoid patients.

Neuregulin-1 (NRG1) is a sensory and motor neuron-derived factor isoform. The isoform SMDF may play a role in motor and sensory neuron development. Neuregulin-1 is a single-pass type I membrane protein normally expressed in the nervous system: spinal cord motor neurons, dorsal root ganglion neurons, and brain. The predominant isoform is expressed in sensory and motor neurons, and highly expressed in developing spinal motor neurons and in developing cranial nerve nuclei. Expression is maintained only in both adult motor neurons and dorsal root ganglion neurons.

Peptidyl-glycine alpha-amidating monooxygenase precursor (PAM) is a bifunctional enzyme that catalyzes 2 sequencial steps in C-terminal alpha-amidation of peptides. The monooxygenase part produces an unstable peptidyl(2-hydroxyglycine) intermediate that is dismutated to glyoxylate and the corresponding desglycine peptide amide by the lyase part. C-terminal amidation of peptides such as neuropeptides is essential for full biological activity. PAM is a single-pass type I membrane protein which can be secreted from secretory granules.

Platelet-derived growth factor A chain precursor (PDGFA) is a potent mitogen for cells of mesenchymal origin. Binding of this growth factor to its affinity receptor elicits a variety of cellular responses. It is released or secreted by platelets upon wounding and plays an important role in stimulating adjacent cells to grow and thereby heals the wound.

Extracellular sulfatase Sulf-2 precursor (SULF-2) exhibits arylsulfatase activity and highly specific endoglucosamine-6-sulfatase activity. It can remove sulfate from the C-6 position of glucosamine within specific subregions of intact heparin. SULF-2 can be localized at the endoplasmic reticulum, Golgi apparatus, Golgi stack, or cell surface. SULF-2 is expressed at highest levels in the ovary, skeletal muscle, stomach, brain, uterus, heart, kidney and placenta.

Slit homolog 2 protein precursor (SLIT2) acts as molecular guidance cue in cellular migration and its function appears to be mediated by interaction with roundabout homolog receptors. SLIT2 seems to be essential for midline guidance in the forebrain by acting as a repulsive signal preventing inappropriate midline crossing by axons projecting from the olfactory bulb. In spinal chord development, SLIT2 may play a role in guiding commissural axons once they reached the floor plate by modulating the response to netrin. In the developing visual system, SLIT2 appears to function as repellent for retinal ganglion axons by providing a repulsion that directs these axons along their appropriate paths prior to, and after passage through, the optic chiasm. It also seems to play a role in branching and arborization of CNS sensory axons, and in neuronal cell migration. SLIT2 is a secreted protein expressed in fetal lung and kidney, and adult spinal cord. Weak expression in adult adrenal gland, thyroid, trachea and other tissues examined was also observed.

The present disclosure provides methods for diagnosing disease states based on the detected presence and/or level of secreted protein isoforms in a biological sample, and/or the detected presence and/or level of biological activity of the secreted protein isoforms. There is reported herein a set of highly significant and biologically relevant secreted protein isoforms that make up a strong signature for ovarian cancer samples. Because these isoforms are secreted, they can be easily detected in the biological fluid of the subject.

The “signature” of an ovarian cancer, as intended herein, consists in the presence of secreted protein isoforms in a sample of a subject experiencing ovarian cancer which is different from the secreted protein isoforms in a sample from a control subject. More specifically, the “signature” is composed of at least one protein isoform, which discriminates between a cancerous tissue and normal tissue with about 90% accuracy. More preferably, the signature is composed of at least 2, 3, 4 or 5 secreted protein isoforms, disclosed for example in Table 1, more preferably at least 6, 7, 8, 9, or 10, preferably 15, 16 or 17. Thus, the combination of more than one secreted protein isoform can also be used as a signature. In an embodiment, the signature comprises a ratio between two protein isoforms (from a given gene) that differs from the ratio determined in a healthy sample. In another embodiment, the signature comprises a protein isoform that is absent (or undetectable) in a healthy sample. Combinations between the two embodiments are also possible.

These detection methods can be carried out in a kit. Thus, the present disclosure further provides kits for detecting the presence and/or a level of a secreted protein isoform in a biological sample and/or the detected presence and/or level of biological activity of the secreted protein isoform. The latter is particularly advantageous when the modulation of alternative splicing that occurs at the onset or during ovarian cancer results in the production of at least two protein isoforms having different biological activity.

Procedures using these kits can be performed by clinical laboratories, experimental laboratories, medical practitioners, or private individuals.

Kits for detecting a secreted protein isoform will comprise a moiety that specifically detects the presence of the protein isoform, either at the polypeptide level or at the mRNA level. In an embodiment, the moiety specifically recognizes to the secreted protein isoform disclosed herein. In a further embodiment, the moiety includes, but is not limited to, an isoform-specific antibody or fragment thereof capable of specifically recognizing the secreted protein isoform. The kits are useful in diagnostic applications. For example, the kit is useful to determine whether a given sample isolated from a subject comprises a secreted protein isoform associated with ovarian cancer. The kits can include one or more specific antibodies (either monoclonal or polyclonal). In yet another embodiment, the kit comprises at least two antibodies (or fragments thereof) each specifically recognizing a different secreted protein isoform. In an additional embodiment, the kit comprises at least two antibodies (or fragments thereof) each specifically recognizing a different secreted protein isoform encoded by the same the same gene. In some embodiments, the antibody specific to a secreted protein isoform is detectably labeled. In other embodiments, the antibody specific to the secreted protein isoform is not labeled; instead, a second, detectably-labeled antibody is provided that binds to the specific antibody. The kit may further include blocking reagents, buffers, and reagents for developing and/or detecting the detectable marker. The kits may further include instructions for use, controls, and interpretive information.

Where the kits provide for detecting the biological (e.g. enzymatic) activity of a secreted protein isoform, it includes a substrate that provides for a detectable product when acted upon by the secreted protein isoform of interest. The kit may further include reagents necessary to detect and develop the detectable marker.

Quantification and detection of secreted protein isoforms disclosed herein can be performed by any means known to those skilled in the art. Means of detection and quantification include but are not limited to precipitation of the protein by an antibody, Western immunoblotting in which the protein is separated by gel electrophoresis, transferred to a suitable support (e.g., nitrocellulose) and visualized by reaction with an antibody(ies); radioimmunoassay, in which the degree to which the protein competes with a radioactively labeled standard for binding to the antibody is used as a means of detecting and quantifying the protein; and enzyme-linked immunosorbent assay (ELISA).

ELISA is a known technique for quantifying proteins in which, generally, an antibody against the protein of interest is immobilized on an inert solid, e.g., polystyrene. A sample to be assayed for the protein of interest is applied to the surface containing immobilized antibody. Protein binds the antibody, forming a complex. This complex is then contacted by a second antibody which binds the same protein and which is covalently bound to an easily assayed enzyme. After washing away any of the second antibody which is unbound, the enzyme in the immobilized complex is assayed, providing a measurement of the amount of protein in the sample. The ELISA procedure can be reversed, i.e., the antigen is immobilized on an inert support (e.g. 96-well microplate) and samples are probed for the presence of antibody to the immobilized antigen.

The biomarkers or secreted protein isoform can also be detected in cells and tissues using immunohistochemical and/or immunofluorescence procedures (such as, for example, FACS analysis).

Secreted protein isoforms can be detected and quantified from samples including, but not limited to, plasma, serum, cerebrospinal fluid, saliva, pleural fluid, peritoneal fluid and amniotic fluid. They can also be detected in tissues or cells obtained from the individual.

The term “sample” as used herein also includes, but is not limited to, blood, bodily fluid, or tissue. The term “bodily fluid” means any fluid produced or secreted within or by a body of a mammal, including human, such as for example blood, lymph, tissue fluid, urine, bile, sweat, synovial fluid, amniotic fluid, abdominal fluid, pericardial fluid, pleural fluid, cerebrospinal fluid, gastric juice, intestinal juice, joint cavity fluid, tears, saliva and nasal discharge.

The present invention will be more readily understood by referring to the following examples which are given to illustrate the invention rather than to limit its scope.

### Example 1

### Mapping Splicing Events by Comprehensive RT-PCR Coverage

A gene list was obtained by a keyword search for “ovarian cancer” in NCBI Gene database. The search was limited to human genes with “known” RefSeq status (Nucleic Acids Res 2005 Jan. 1; 33(1): D501-D504). The genes generated from this search were cross referenced with the AceView™ database. The exon structure of each gene was determined using AceView as a source for cDNAs and multi-exon ESTs (Thierry-Mieg & Thierry-Mieg, 2006, Genome Biol, 7 Suppl 1, S12, 1-14). The LISA platform identifies all splice sites and generates a splicing map. Concurrently, the LISA plateform applies a modified Primer-3-based (Rozen & Skaletsky, 2000, Methods Mol Biol, 132: 365-386) algorithm for the automated design of PCR primers. Each gene's AceView transcript set was mapped into the LISA platform database and the LISA platform design module was used to generate a PCR experiment set. This module is a perl script which reads input sequences from the database and automatically designs PCR primers to characterize the exon structure of the gene. The overall strategy allowed designing primers for all exons in the transcript set, such that PCR experiments flanking all possible exon-exon junctions could be designed. In practice, a forward and reverse primer was designed for all internal exons, and single primers were designed for terminal exons. Primers were synthesized in 96-well plates on a 25 nmole scale (IDT, Coralville, Iowa) (see Table 3 for list of primers). PCR reactions were formulated to cover all constitutive splicing events with a single reaction and alternative splicing events were covered by at least two independent reactions.

Reverse transcription was performed on 2 μg total RNA samples in the presence of RNase inhibitor according to the manufacturers' protocols. Reactions were primed with both (dT)21 and random hexamers at final concentrations of 1 and 0.9 umol/L, respectively. The integrity of the cDNA was assessed by SYBR green-based quantitative PCR, done on three housekeeping genes: MRPL19, PUM1, and GAPDH (primer sequences available on request). Ct values for these genes, typically in the range of 14 to 25, depending on the gene, were used to verify the integrity of each cDNA sample. Following qPCR, the samples were analyzed by capillary electrophoresis to ensure that only one amplicon of the expected size was obtained. End-point PCR reactions were done on 20 ng cDNA in 10 uL final volume containing 0.2 mmol/L each dNTP, 1.5 mmol/L MgCl2, 0.6 umol/L each primer, and 0.2 units of Taq DNA polymerase. An initial incubation of 2 min at 95° C. was followed by 35 cycles at 94° C. 30 s, 55° C. 30 s, and 72° C. 60 s. The amplification was completed by a 2 min incubation at 72° C. Relative quantification using this method was verified for six alternative splicing events by comparison with isoform-specific quantitative PCR, and was found to be in strong agreement, to within 5% in all cases. PCR reactions are carried out using a liquid handling system linked to thermocyclers, and the amplified products were analyzed by automated chip-based microcapillary electrophoresis on Caliper LC-90® instruments (Caliper LifeSciences). Amplicon sizing and relative quantification was performed by the manufacturer's software, before being uploaded to the LISA database.

TABLE 3
List of primer pairs for each identified secreted splice variant proteins and
listed in the sequence listing.

| Name of | | |
|---|---|---|
| biomarker | Forward primer | Reverse primer |
| APP | TGAGGAACCCTACGAAGAAGC | CGAGATACTTGTCAACGGCA |
| | (SEQ ID NO: 1) | (SEQ ID NO: 2) |
| BSG | TTTCAACCTCCAAGAGACGC | AAGACGCAGGAGTACTCTCCC |
| | (SEQ ID NO: 3) | (SEQ ID NO: 4) |
| BTC | ACCACCACACAATCAAAGCG | TTACGACGTTTCCGAAGAGG |
| | (SEQ ID NO: 5) | (SEQ ID NO: 6) |
| CD97 | TGGACGAATGTCAGCAGAAC | TGTATGAACCCACGGTGTTG |
| | (SEQ ID NO: 7) | (SEQ ID NO: 8) |
| CEACAM1 | GGCATTGTGATTGGAGTAGTGG | CTTGTTAGGTGGGTCATTGGAG |
| | (SEQ ID NO: 9) | (SEQ ID NO: 10) |
| CLSTN1 | CACAGAGAACGACAACACCG | CGAATGACTCCCTCACCAGT |
| | (SEQ ID NO: 11) | (SEQ ID NO: 12) |
| CMTM1 (or CKLF) | TTCGCAGAACCTACTCAGGC | GCCAACACAGATACGATGAGC |
| | (SEQ ID NO: 13) | (SEQ ID NO: 14) |
| F3 | CTCGGACAGCCAACAATTCA | CCACTCCTGCCTTTCTACACTT |
| | (SEQ ID NO: 15) | (SEQ ID NO: 16) |
| IGSF4 | CACCACCATCCTTACCATCATC | AGAATGATGAGCAAGCACAGC |
| | (SEQ ID NO: 17) | (SEQ ID NO: 18) |
| KITLG | CATGATAACCCTCAAATATGTCCC | GCCCAGTGTAGGCTGGAGT |
| | (SEQ ID NO: 19) | (SEQ ID NO: 20) |
| LGALS9 | GTGATGGTGAACGGGATCCT | GTTGGCAGGCCACACGCC |
| | (SEQ ID NO: 21) | (SEQ ID NO: 22) |
| MMP19 | AGCCAGAAGATATCACCGAGG | TCAGTCCAGAACTCGTCTTCG |
| | (SEQ ID NO: 23) | (SEQ ID NO: 24) |
| NRG1 | GTTTACTGGTGATCGCTGCC | TGGGCTGTGGAAGTATAGTGAC |
| | (SEQ ID NO: 25) | (SEQ ID NO: 26) |
| PAM | CTACAGCAGCCAAAACGAGAAG | CCTGGCCTGGTAACAAGTATACTC |
| | (SEQ ID NO: 27) | (SEQ ID NO: 28) |
| PDGFA | GTGAGGTTAGAGGAGCATTTGG | CAGGAATGTAACACGCCATG |
| | (SEQ ID NO: 29) | (SEQ ID NO: 30) |
| SLIT2 | CTGGCAAACAAAAGAATTGGAC | AGATCCGCAAAGCAGTCTCC |
| | (SEQ ID NO: 31) | (SEQ ID NO: 32) |
| SULF2 | ATGTCCTCAACCAGCTACACG | CTTAACCTTCCCAGCCTTCC |
| | (SEQ ID NO: 33) | (SEQ ID NO: 34) |

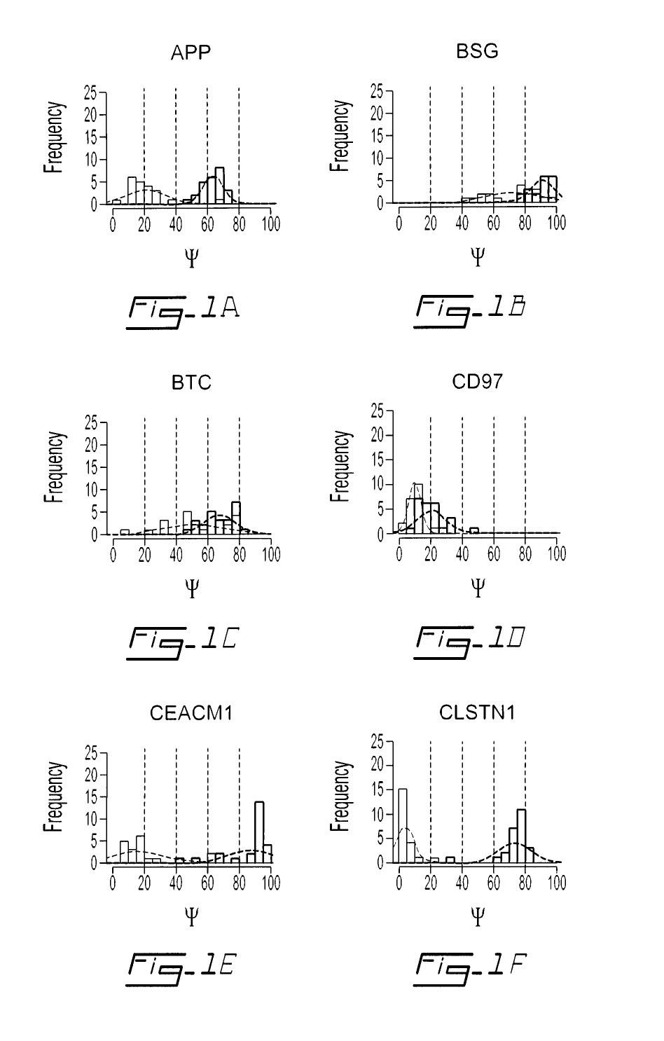

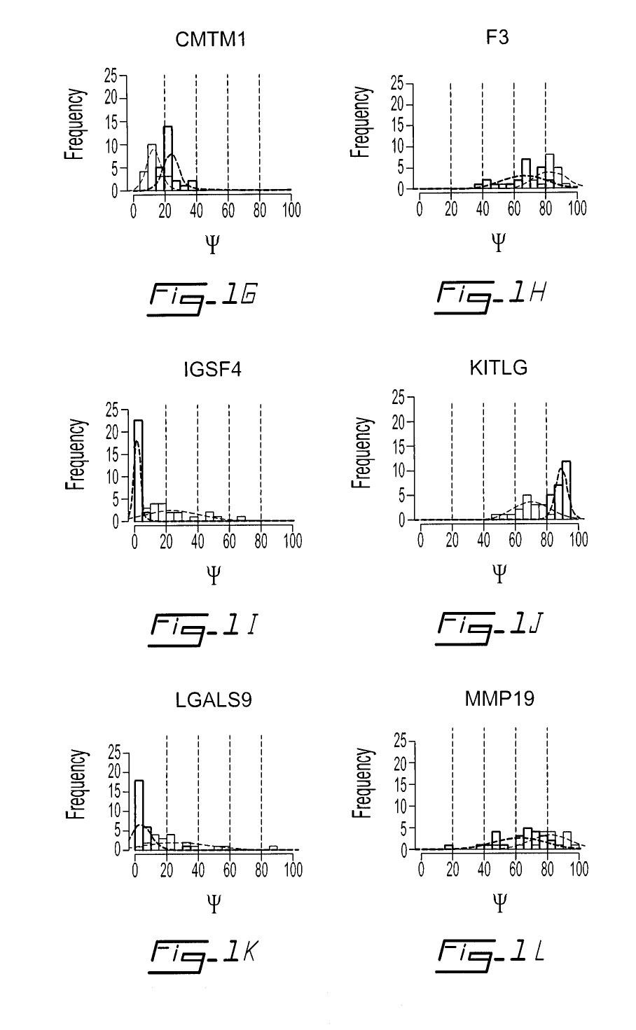

The splicing patterns observed for seventeen different protein isoform are shown in FIG. 1.

Once alternative splicing patterns were determined, an implemented an improved secreted protein prediction approach was used to select from the alternative splicing patterns identified which correspond to secreted splice variants. Alternative splicing events occurring in secreted or transmembrane proteins were selected by fetching data from the Uniprot database and using keyword search “secreted or transmembrane”.

### Example 2

### Detection of Secreted Splice Variant in a Patient Sample

The blood samples were collected from patients and normal control donors using Vacutainer® K2-EDTA. As soon as possible, the tubes were centrifuged for 15 minutes at 2000 g (room temperature) in Beckman Coulter® table centrifuge (Allegra® 25R). The upper phases were then collected, aliquoted and stored at −80° C.

IMUBIND® Tissue Factor Elisa kit from American Diagnostica Inc was used. The following materials are provided in the kit: pre-coated micro-test strips (with capture antibody), standard proteins (F3), detection antibody, enzyme conjugate diluent, wash buffer and sample buffer. All reagents were prepared as recommended by the manufacturer. 100 μl of tissue factor standards (ranging 50 to 1000 pg/ml) or diluted plasma samples (1:4) (25 μl in 100 μl of sample buffer) were added to pre-coated micro-test wells, covered with lid and incubated overnight at 4° C. Following four washes (200 μl per well) with wash buffer, 100 μl of detection antibody were added to each well and incubated at room temperature for 1 hour. After four washes, 100 μl of diluted enzyme conjugate (1:1000) were added and incubated as previously, then the wells were washed again. Colorimetric detection was performed by adding 100 μl of substrate solution for 20 minutes at room temperature until blue coloration appeared, then stopped by addition of 50 μl of 0.5 M H₂SO₄. Absorbance monitoring at 450 nm were done on Power Wave XS™ microplate reader (BioTek). Blank background averages were deducted from all standards and sample readings.

Unknown samples values were interpolated from the tissue factor standard curve, then corrected for the dilution factor used.

The ranges of the obtained concentrations were in agreement with previous reports (Förster et al, 2006, Clin Chim Acta., 364(1-2):12-21; Albrecht et al., 1996, Thromb Haemost., 75(5):772-7).

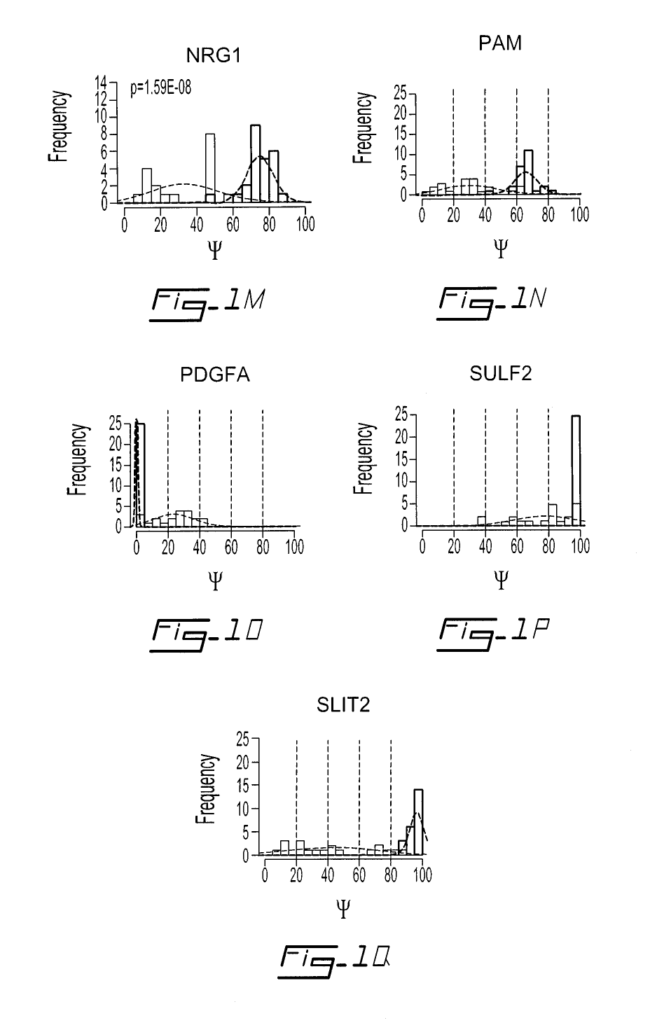

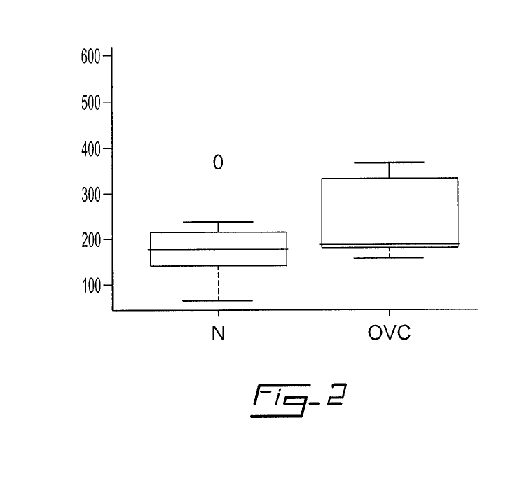

As shown in FIG. 2, the F3 protein is expressed at higher levels in the sera of a patient having ovarian cancer when compared to the levels of the F3 protein in the sera of a normal patient (who is not experiencing ovarian cancer).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for diagnosis or prognosis of ovarian cancer in a subject by detecting a signature of at least one secreted protein isoform comprising the steps of:
a) obtaining a sample from said subject, and
b) determining whether the sample from step a) contains the signature specific to ovarian cancer,
wherein the presence of the signature is indicative of the presence of ovarian cancer in the individual or the progression of ovarian cancer in the individual.

2. The method of claim 1, wherein said signature comprises at least two secreted protein isoforms.

3. The method of claim 1, wherein the at least one secreted protein isoform is at least one of amyloid beta A4 protein precursor (APP), basigin precursor (BSG), probetacellulin precursor (BTC), CD97 antigen precursor (CD97), Carcinoembryonic antigen-related cell adhesion molecule 1 precursor (CEACAM1), Calsyntenin-1 precursor (CLSTN1), Chemokine-like factor (CMTM1 or CKLF), Tissue factor precursor (F3), Cell adhesion molecule 1 precursor (IGSF4), Kit ligand precursor (KITLG), Galectin-9 (LGSF9), Matrix metalloproteinase-19 precursor (MMP19), Neuregulin-1 (NRG1), Peptidyl-glycine alpha-amidating monooxygenase precursor (PAM), Platelet-derived growth factor A chain precursor (PDGFA), Extracellular sulfatase Sulf-2 precursor (SULF-2), or Slit homolog 2 protein precursor (SLIT2).

4. The method of claim 3, wherein the APP secreted splice variant is encoded by the nucleotide sequence set forth in SEQ ID NO:35.

5. The method of claim 3, wherein the BSG secreted splice variant is encoded by the nucleotide sequence set forth in SEQ ID NO:36.

6. The method of claim 3, wherein the BTC secreted splice variant is encoded by the nucleotide sequence set forth in SEQ ID NO:37.

7. The method of claim 3, wherein the CD97 secreted splice variant is encoded by the nucleotide sequence set forth in SEQ ID NO:38.

8. The method of claim 3, wherein the CEACAM1 secreted splice variant is encoded by the nucleotide sequence set forth in SEQ ID NO:39.

9. The method of claim 3, wherein the CLSTN1 secreted splice variant is encoded by the nucleotide sequence set forth in SEQ ID NO:40.

10. The method of claim 3, wherein the CMTM1 or CKLF secreted splice variant is encoded by the nucleotide sequence set forth in SEQ ID NO:41.

11. The method of claim 3, wherein the F3 secreted splice variant is encoded by the nucleotide sequence set forth in SEQ ID NO:42.

12. The method of claim 3, wherein the IGSF4 secreted splice variant is encoded by the nucleotide sequence set forth in SEQ ID NO:43.

13. The method of claim 3, wherein the KITLG secreted splice variant is encoded by the nucleotide sequence set forth in SEQ ID NO:44.

14. The method of claim 3, wherein the LGSF9 secreted splice variant is encoded by the nucleotide sequence set forth in SEQ ID NO:45.

15. The method of claim 3, wherein the MMP19 secreted splice variant is encoded by the nucleotide sequence set forth in SEQ ID NO:46.

16. The method of claim 3, wherein the NRG1 secreted splice variant is encoded by the nucleotide sequence set forth in SEQ ID NO:47.

17. The method of claim 3, wherein the PAM secreted splice variant is encoded by the nucleotide sequence set forth in SEQ ID NO:48.

18. The method of claim 3, wherein the PDGFA secreted splice variant is encoded by the nucleotide sequence set forth in SEQ ID NO:49.

19. The method of claim 3, wherein the SLIT2 secreted splice variant is encoded by the nucleotide sequence set forth in SEQ ID NO:50.

20. The method of claim 3, wherein the SULF-2 secreted splice variant is encoded by the nucleotide sequence set forth in SEQ ID NO: 51.

21. The method of claim 1, wherein said detecting comprises contacting an antibody specific for the at least one protein isoform with said sample and detecting the presence of a complex between the antibody and the at least one protein isoform.

22. A diagnostic or prognostic kit for ovarian cancer for detecting a signature of at least one secreted protein isoform in a sample of a subject, said kit comprising:
a) a moiety that specifically recognized the at least one secreted protein isoform or a mRNA encoding the at least one secreted protein isoform of the signature; and
b) a set of instructions for using said moiety to detect the signature specific to ovarian cancer.

23. The kit of claim 22, further comprising means to detect the complex between the moiety and the at least one protein isoform or the mRNA.

24. The kit of claim 22, further comprising a blocking reagent or buffer.

25. The kit of claim 22, wherein said moiety is an antibody or fragment thereof specific for the at least one secreted protein isoform.

26. The kit of claim 22, wherein said signature comprises at least two secreted protein isoforms.

27. The kit of claim 22, wherein the at least one secreted protein isoform is at least one of amyloid beta A4 protein precursor (APP), basigin precursor (BSG), probetacellulin precursor (BTC), CD97 antigen precursor (CD97), Carcinoembryonic antigen-related cell adhesion molecule 1 precursor (CEACAM1), Calsyntenin-1 precursor (CLSTN1), Chemokine-like factor (CMTM1 or CKLF), Tissue factor precursor (F3), Cell adhesion molecule 1 precursor (IGSF4), Kit ligand precursor (KITLG), Galectin-9 (LGSF9), Matrix metalloproteinase-19 precursor (MMP19), Neuregulin-1 (NRG1), Peptidyl-glycine alpha-amidating monooxygenase precursor (PAM), Platelet-derived growth factor A chain precursor (PDGFA), Extracellular sulfatase Sulf-2 precursor (SULF-2), or Slit homolog 2 protein precursor (SLIT2).

28. The kit of claim 27, wherein the APP secreted splice variant is encoded by the nucleotide sequence set forth in SEQ ID NO:35.

29. The kit of claim 27, wherein the BSG secreted splice variant is encoded by the nucleotide sequence set forth in SEQ ID NO:36.

30. The kit of claim 27, wherein the BTC secreted splice variant is encoded by the nucleotide sequence set forth in SEQ ID NO:37.

31. The kit of claim 27, wherein the CD97 secreted splice variant is encoded by the nucleotide sequence set forth in SEQ ID NO:38.

32. The kit of claim 27, wherein the CEACAM1 secreted splice variant is encoded by the nucleotide sequence set forth in SEQ ID NO:39.

33. The kit of claim 27, wherein the CLSTN1 secreted splice variant is encoded by the nucleotide sequence set forth in SEQ ID NO:40.

34. The kit of claim 27, wherein the CMTM1 or CKLF secreted splice variant is encoded by the nucleotide sequence set forth in SEQ ID NO:41.

35. The kit of claim 27, wherein the F3 secreted splice variant is encoded by the nucleotide sequence set forth in SEQ ID NO:42.

36. The kit of claim 27, wherein the IGSF4 secreted splice variant is encoded by the nucleotide sequence set forth in SEQ ID NO:43.

37. The kit of claim 27, wherein the KITLG secreted splice variant is encoded by the nucleotide sequence set forth in SEQ ID NO:44.

38. The kit of claim 27, wherein the LGSF9 secreted splice variant is encoded by the nucleotide sequence set forth in SEQ ID NO:45.

39. The kit of claim 27, wherein the MMP19 secreted splice variant is encoded by the nucleotide sequence set forth in SEQ ID NO:46.

40. The kit of claim 27, wherein the NRG1 secreted splice variant is encoded by the nucleotide sequence set forth in SEQ ID NO:47.

41. The kit of claim 27, wherein the PAM secreted splice variant is encoded by the nucleotide sequence set forth in SEQ ID NO:48.

42. The kit of claim 27, wherein the PDGFA secreted splice variant is encoded by the nucleotide sequence set forth in SEQ ID NO:49.

43. The kit of claim 27, wherein the SLIT2 secreted splice variant is encoded by the nucleotide sequence set forth in SEQ ID NO:50.

45. The kit of claim 27, wherein the SULF-2 secreted splice variant is encoded by the nucleotide sequence set forth in SEQ ID NO: 51.

45. A method for profiling an ovarian cancer in a subject by detecting a signature of at least one secreted protein isoform comprising the steps of:
a) obtaining a sample from said subject, and
b) determining whether the sample from step a) contains the signature specific to ovarian cancer.

46. The method of claim 45, wherein said signature comprises at least two secreted protein isoforms.

47. The method of claim 45, wherein the at least one secreted protein isoform is at least one of amyloid beta A4 protein precursor (APP), basigin precursor (BSG), probetacellulin precursor (BTC), CD97 antigen precursor (CD97), Carcinoembryonic antigen-related cell adhesion molecule 1 precursor (CEACAM1), Calsyntenin-1 precursor (CLSTN1), Chemokine-like factor (CMTM1 or CKLF), Tissue factor precursor (F3), Cell adhesion molecule 1 precursor (IGSF4), Kit ligand precursor (KITLG), Galectin-9 (LGSF9), Matrix metalloproteinase-19 precursor (MMP19), Neuregulin-1 (NRG1), Peptidyl-glycine alpha-amidating monooxygenase precursor (PAM), Platelet-derived growth factor A chain precursor (PDGFA), Extracellular sulfatase Sulf-2 precursor (SULF-2), or Slit homolog 2 protein precursor (SLIT2).

48. The method of claim 47, wherein the APP secreted splice variant is encoded by the nucleotide sequence set forth in SEQ ID NO:35.

49. The method of claim 47, wherein the BSG secreted splice variant is encoded by the nucleotide sequence set forth in SEQ ID NO:36.

50. The method of claim 47, wherein the BTC secreted splice variant is encoded by the nucleotide sequence set forth in SEQ ID NO:37.

51. The method of claim 47, wherein the CD97 secreted splice variant is encoded by the nucleotide sequence set forth in SEQ ID NO:38.

52. The method of claim 47, wherein the CEACAM1 secreted splice variant is encoded by the nucleotide sequence set forth in SEQ ID NO:39.

53. The method of claim 47, wherein the CLSTN1 secreted splice variant is encoded by the nucleotide sequence set forth in SEQ ID NO:40.

54. The method of claim 47, wherein the CMTM1 or CKLF secreted splice variant is encoded by the nucleotide sequence set forth in SEQ ID NO:41.

55. The method of claim 47, wherein the F3 secreted splice variant is encoded by the nucleotide sequence set forth in SEQ ID NO:42.

56. The method of claim 47, wherein the IGSF4 secreted splice variant is encoded by the nucleotide sequence set forth in SEQ ID NO:43.

57. The method of claim 47, wherein the KITLG secreted splice variant is encoded by the nucleotide sequence set forth in SEQ ID NO:44.

58. The method of claim 47, wherein the LGSF9 secreted splice variant is encoded by the nucleotide sequence set forth in SEQ ID NO:45.

59. The method of claim 47, wherein the MMP19 secreted splice variant is encoded by the nucleotide sequence set forth in SEQ ID NO:46.

60. The method of claim 47, wherein the NRG1 secreted splice variant is encoded by the nucleotide sequence set forth in SEQ ID NO:47.

61. The method of claim 47, wherein the PAM secreted splice variant is encoded by the nucleotide sequence set forth in SEQ ID NO:48.

62. The method of claim 47, wherein the PDGFA secreted splice variant is encoded by the nucleotide sequence set forth in SEQ ID NO:49.

63. The method of claim 47, wherein the SLIT2 secreted splice variant is encoded by the nucleotide sequence set forth in SEQ ID NO:50.

64. The method of claim 47, wherein the SULF-2 secreted splice variant is encoded by the nucleotide sequence set forth in SEQ ID NO: 51.
